(19) United States
(12) Patent Application Publication
(10) Pub. No.: US 2024/0032890 A1
(43) Pub. Date: Feb. 1, 2024

(54) GESTATIONAL AGE ESTIMATION METHOD AND APPARATUS

(71) Applicant: Intelligent Ultrasound Limited, Cardiff (GB)

(72) Inventors: Nicholas Sleep, Cardiff (GB); Martin Benson, Cardiff (GB); Aris Papageorghiou, Cardiff (GB)

(73) Assignee: Intelligent Ultrasound Limited, Cardiff (GB)

(21) Appl. No.: 18/359,444

(22) Filed: Jul. 26, 2023

(30) Foreign Application Priority Data
Jul. 28, 2022 (GB) 2211036.5

(51) Int. Cl. A61B 8/08; G06T 7/00; G06T 7/62

(52) U.S. Cl. CPC A61B 8/0866; A61B 8/5223; G06T 7/0012; G06T 7/62; G06T 2207/20084; G06T 2207/30044

(57) ABSTRACT

A method of estimating gestational age (GA) is shown generally at 3000. At Step 3010 an ultrasound image is acquired. The image is fed into the model at Step 3020 and a predicted GA value, together with an associated confidence interval are calculated at Step 3030. At Step 3040 it is determined whether the confidence interval is narrower than a value for the best confidence interval obtained. If not, the ultrasound operation is optionally directed to acquire an ultrasound image of a particular plane in the fetus at Step 3050. On the other hand, if the value is determined to be narrower than the best obtained at Step 3040, the predicted value and confidence interval are displayed at Step 3060 and a register for the value of the best confidence interval is updated at Step 3070.

The present invention relates to a method and apparatus for estimating the gestational age of a fetus, and optionally for assessing some other key aspects of pregnancy evaluation.

Gestational Age (GA) of a fetus is defined as the time elapsed since the start of the Last Menstrual Period (LMP). Knowing GA has numerous clinical applications, including due date estimation, and growth surveillance. While in some cases it may be assessed by the mother's recollection of their last period date, doing so is problematic in general and so estimating it from ultrasound data is common practice.

An estimate of fetal gestational age is therefore a key output of routine obstetric scanning and is obtained by taking certain measurements of the fetus. In early pregnancy, this is currently done by measuring Crown Rump Length (CRL), and later it is estimated from a number of measurements, commonly including Head Circumference, Femur Length and Abdominal Circumference.

Measuring CRL is therefore a key component of current routine first trimester scanning. It requires capturing an image in the correct imaging plane which is defined by certain properties that are well-known to trained sonographers and defined in various imaging standards internationally, the key ones being that:
the crown and rump of the fetus are visible
flexion of the fetal neck is “neutral”
The fetus is in a horizontal position in relation to the ultrasound beam

In practice, obtaining such an image is time-consuming, with much time being wasted on waiting for the fetus to be in a suitable position for measurement, and there is a clinical desire for a more time-efficient method of GA estimation. It has been estimated that around a third of the total first trimester scanning time is spent on obtaining a suitable CRL image.

A number of other key clinical assessments are made during fetal screening alongside assessment of GA. While the detail of these will vary across territories and scanning institutions, the following are often assessed: The presence of multiple fetuses, and in this case their chorionicity and amnionicity; demonstrating fetal viability by confirming cardiac activity and estimating the heart rate of the fetus(es); whether the pregnancy is ectopic; presence of fetal activity; risk of trisomy in the fetus(es); estimated fetal weight and body composition; fetal growth and development including of the brain; presence of pathologies such as acrania, gastroschisis, spina bifida or an anterior wall defect; presence of markers for genetic abnormality such as increased nuchal fold thickness or nasal bone hypoplasia; presence of risk factors for poor outcomes such as stillbirth; the sex of the fetus(es); fetal presentation (e.g. breech, cephalic etc.); placental location; categorisation of placental appearance; estimating the amount of amniotic fluid.

Improved methods for assessing these fetal characteristics are therefore also desirable.

Embodiments of the present invention aim to provide a method for obtaining reliable GA estimates throughout the course of gestation that does not require such stringent imaging criteria as has been necessary in previously considered approaches. Further embodiments include assessment of other key fetal characteristics including, for example, those listed above.

The present invention is defined in the attached independent claims, to which reference should now be made. Further, preferred features may be found in the sub-claims appended thereto.

According to one aspect of the present invention, there is provided a method for estimating the gestational age of a fetus, the method comprising obtaining at least one ultrasound image of at least a part of the fetus, and calculating an estimate of the gestational age of the fetus, and a corresponding confidence assessment for the estimate, from the image .

The method preferably comprises calculating the gestational age of the fetus and an associated confidence interval from any 2D ultrasound image of the fetus. This is in contrast to previously considered methods which require either 2D images from very specific planes to be acquired, or otherwise require a 3D volume.

The method may comprise calculating the gestational age of the fetus from an ultrasound image obtained at any point during gestation.

In a preferred arrangement, the method does not require an ultrasound machine operator to observe the or each ultrasound image.

Preferably, the method comprises calculating the estimate of the gestational age and/or the corresponding confidence assessment for the estimate using a trained machine learning model.

Preferably, the machine learning model is produced by training it on a set of representative ultrasound (US) images of fetuses, for each of which the GA at the time of imaging is known. Model training is preferably accomplished via a process known as supervised learning (which will be well known to a person skilled in the art), whereby the model is configured to achieve the strongest possible (whilst also robust and generalisable) association between each image and its corresponding GA value. In the present invention, the model and supervised training method are preferably additionally configured to output a range of values in which the GA is expected to lie. The known GA values preferably fall outside of this range with low probability.

Preferably, the supervised machine learning process takes the form of deep learning, in which the model is an artificial neural network. The supervised learning method may consist of optimising the parameters of this network, via stochastic gradient descent, in order to minimise a loss function. Preferably, the loss function shall be constructed such that for regression tasks (including for example estimation of GA and fetal heart rate) it generates improved performance in terms of both the accuracy of the estimate and the likelihood that the range supplied contains the true value. There now follows, by way of example, a class of loss functions which have this property. The description is framed in terms of GA prediction, but the skilled person will recognise that it is operable for other regression tasks.

### Denote:

x=an ultrasound image of at least part of a fetus
y=the GA of the fetus
[symbol] , [symbol] =Two outputs of the neural network, representing respectively the predicted GA and the width of a confidence interval around it

A class of functions that have the desired properties are described by

L=L_{reg}+αL_{conf}

### Where:

L_{reg} is any non-decreasing function of any semi-norm applied to y− [symbol] (or alternatively of ln y− [symbol] if the prediction task is performed in log-space)
L_{conf} is any function having the following properties:
   For some non-decreasing function f: [symbol] → [symbol] ⁺, and for some function g which is a non-decreasing function of a semi-norm applied to y− [symbol] (or ln y− [symbol] ), and for some constant c ∈

[symbol]

$${L}_{conf} = \{ \begin{matrix}c & if g ( y , ) < f ( ) \\ c + g ( y , ) - f ( ) & otherwise\end{matrix}$$

α is an algorithm hyperparameter

In a preferred arrangement, the loss function is defined as:

L=(ln y− [symbol] )²+α[max(0, |ln y− [symbol] |−f₊( [symbol] ))]

### Where:

   |x| is used to denote the absolute value of x
f₊denotes the softplus function f₊(x)=ln(1+e^{x})
α is an algorithm hyperparameter

For classification tasks (including, for example, prediction of: chorionicity and amnionicity; the sex of the fetus(es); whether the pregnancy is ectopic; presence of fetal activity; risk of trisomy in the fetus(es); presence of pathologies such as acrania or gastroschisis; fetal presentation; placental location) it is also desirable that the model should both generate a prediction and quantify the uncertainty inherent in it. For such tasks, preferably, the loss function shall be constructed such that it generates improved performance in terms of both the accuracy of the estimate and quantification of the uncertainty inherent in it. We now describe, by way of example, a loss function which has this property. The description is framed in terms of predicting if the pregnancy is singleton or multiple, but the skilled person will recognise that it is readily operable for other categorical classification tasks.

### Denote:

x=an ultrasound image of at least part of the content of a uterus containing one or more fetuses
y=A one-hot encoded variable indicating singleton or multiple pregnancy
K (=2 in this case) the number of categories in the dependent variable
α=[α₁, . . . , α_{K}]∈( [symbol] ⁺)^{K} the output of the neural network, representing concentration parameters of a Dirichlet distribution
Γ(·) is the well-known Gamma function

$$ψ ( x ) = \frac{d}{d x} ln ( Γ ( x ) )$$

is the digamma function

The loss (for a single example) is defined as:

L=L_{class}+αL_{conf}

### Where:

$${L}_{class} = {\sum }_{i = 1}^{K} {y}_{i} [ ln ( {\sum }_{j = 1}^{K} {α}_{j} ) - ln ( {α}_{i} ) ]$$

$${\tilde{α}}_{ι} = {y}_{i} + ( 1 - {y}_{i} ) {α}_{i} ∀ i ∈ \{ 1 , ⋯ , K \}$$

$${L}_{conf} = ln ( \frac{Γ ( {\sum }_{i = 1}^{K} \tilde{{α}_{ι}} )}{Γ ( K ) {\prod }_{i = 1}^{K} Γ ( \tilde{{α}_{ι}} )} ) + {\sum }_{i = 1}^{K} ( \tilde{{α}_{ι}} - 1 ) ( ψ ( \tilde{{α}_{ι}} ) - ψ ( {\sum }_{j = 1}^{K} \tilde{{α}_{j}} ) )$$

and α is an algorithm hyperparameter.

The loss is, of course, summed over all examples in the dataset. The skilled person will recognise that many other loss formulations may be used to achieve the same objective, including formulations based on mean squared error or Bayes risk, for example, and also ones based on distributions other than Dirichlet.

Having trained such a model it is possible to obtain from it point estimates of the class probabilities by computing, for example, the expectation of the resultant Dirichlet distribution:

$${p}_{i} = \frac{{α}_{i}}{{\sum }_{j = 1}^{K} {α}_{j}} ∀ i ∈ \{ 1 , ⋯ , K \}$$

A predicted category may be obtained by identifying the category having the highest class probability:

$${i}^{*} = {arg max}_{i ∈ \{ 1 , ⋯ , K \}} {p}_{i}$$

The uncertainty inherent in the predicted category may be estimated, for example, by computing the variance associated with it:

$$Var ( {X}_{{i}^{*}} ) = \frac{{p}_{{i}^{*}} ( 1 - {p}_{{i}^{*}} )}{1 + {\sum }_{j = 1}^{K} {α}_{j}}$$

Preferably the neural network architecture will be a convolutional neural network, a vision transformer, or a variant thereof. Preferably the stochastic gradient descent will be performed using the Adam optimiser.

In a preferred arrangement, the method comprises determining at least one relative dimension of one or more anatomical features of the fetus.

The method preferably comprises determining a confidence value of the estimate of the gestational age. The confidence value may comprise an expression of the confidence that the estimated gestational age of the fetus is accurate. The method may comprise determining a confidence value of the estimate of the gestational age expressed as a range of ages, wherein the narrower the range the higher the confidence value.

The method may comprise obtaining a plurality of ultrasound images of the fetus. In a preferred arrangement, the method comprises determining a GA estimate and corresponding confidence range by applying a machine learning model to a plurality of ultrasound images, then applying numerical processing to the plurality of outputs obtained (including, though not limited to, averaging them).

In a preferred arrangement, one or more of the steps of determining a relative dimension, calculating a gestational age estimate and determining a confidence value is performed by an electronic processor.

Preferably, the step of determining at least one relative dimension within the image comprises determining a scale of one part of the image relative to at least one other part of the same image. Alternatively, or in addition, the step of determining at least one relative dimension within the image may comprise determining a scale of one part of the image relative to at least one part of at least one other image.

The image (or each of the images) may comprise an image of at least one anatomical feature of a fetus.

The method may comprise producing a plurality of estimates, more preferably with a plurality of corresponding confidence values, and filtering the plurality of estimates to select only one or more estimates that meet a predetermined confidence value threshold. The method may comprise producing a plurality of estimates, more preferably with a plurality of corresponding confidence values, and ranking the estimates based on confidence value.

The method may include directing an operative to obtain one or more specific images of the fetus. In one example, the operative may be instructed from the outset to acquire images of certain regions of the fetus that are known (whether through reasoning derived from clinical expertise or via data analysis) to yield accurate estimates of the GA. In another example the operative may be dynamically instructed, while scanning, to acquire additional images in the event that the images already acquired have wide confidence ranges associated with them.

The method may include arithmetically processing, for example averaging, the estimates of gestational age obtained from a plurality of images.

The images may be selected by an operative during a scan. Alternatively, or in addition, the images may comprise one or more sample frames captured/obtained automatically during a scanning operation.

The method may comprise a method of estimating biometric measurement(s) including, for example, one or more of crown rump length (CRL), head circumference (HC), femur length (FL), abdominal circumference (AC) and trans-cerebellar diameter (TCD).

The invention also includes a method of making one or more clinical assessments of a fetus, the method comprising obtaining an ultrasound image of the fetus and processing the image using a trained machine learning model to determine one or more of the following, including but not limited to: the presence of multiple fetuses, and in this case their chorionicity and amnionicity; demonstrating fetal viability by confirming cardiac activity and estimating the heart rate of the fetus(es); whether the pregnancy is ectopic; presence of fetal activity; risk of trisomy in the fetus(es); estimated fetal weight and body composition; fetal growth and development including of the brain; presence of pathologies such as acrania, gastroschisis, spina bifida or an anterior wall defect; presence of markers for genetic abnormality such as increased nuchal fold thickness or nasal bone hypoplasia; presence of risk factors for poor outcomes such as stillbirth; the sex of the fetus(es); fetal presentation (e.g. breech, cephalic etc.); placental location; categorisation of placental appearance; estimating the amount of amniotic fluid.

According to another aspect of the present invention, there is provided apparatus for estimating the gestational age of a fetus, the apparatus comprising an ultrasound image capturing device for obtaining an image of at least a part of the fetus, and an electronic processing device arranged to process the image, and to calculate an estimate of the gestational age of the fetus, and a corresponding confidence assessment of the estimate.

Preferably, the processing device is arranged to use a trained machine learning model to calculate the estimate and/or the confidence assessment.

The apparatus is preferably arranged to capture and process a plurality of ultrasound images of the fetus. In a preferred arrangement, the apparatus is arranged to determine at least one relative dimension of one or more anatomical features of the fetus.

The apparatus is preferably arranged to determine a confidence value for the, or each, estimate of the gestational age. The confidence value may comprise an expression of the confidence that the estimated gestational age of the fetus is accurate. The apparatus is preferably arranged in use to determine a confidence value of the estimate of the gestational age expressed as a range of ages, wherein the narrower a range the higher the confidence value.

The apparatus may be configured to provide real-time feedback to the user on the confidence range of the GA estimate that has been obtained, in order that they may direct scanning toward more suitable images when necessary.

The processing may be implemented directly on the ultrasound apparatus or else on separate hardware which receives a video feed from the ultrasound apparatus and, optionally displays results on a separate monitor.

The image (or each of the images) may comprise an image of at least one anatomical feature of a fetus.

The invention also includes apparatus for making one or more clinical assessments of a fetus, the apparatus comprising a processor for processing the image using a trained machine learning model to determine one or more of the following, including but not limited to: the presence of multiple fetuses, and in this case their chorionicity and amnionicity; demonstrating fetal viability by confirming cardiac activity and estimating the heart rate of the fetus(es); whether the pregnancy is ectopic; presence of fetal activity; risk of trisomy in the fetus(es); estimated fetal weight and body composition; fetal growth and development including of the brain; presence of pathologies such as acrania, gastroschisis, spina bifida or an anterior wall defect; presence of markers for genetic abnormality such as increased nuchal fold thickness or nasal bone hypoplasia; presence of risk factors for poor outcomes such as stillbirth; the sex of the fetus(es); fetal presentation (e.g. breech, cephalic etc.); placental location; categorisation of placental appearance; estimating the amount of amniotic fluid.

In a further aspect, the invention provides a computer programme product on a computer readable medium, comprising instructions that, when executed by a computer, cause the computer to perform a method of estimating the gestational age of a fetus, or for making one or more clinical assessments of a fetus, the method being according to any statement herein.

The invention also comprises a program for causing a device to perform a method of estimating the gestational age of a fetus, or for making one or more clinical assessments of a fetus, according to any statement herein.

The invention may include any combination of the features or limitations referred to herein, except such a combination of features as are mutually exclusive, or mutually inconsistent.

A preferred embodiment of the present invention will now be described, by way of example only, with reference to the accompanying diagrammatic drawings, in which:

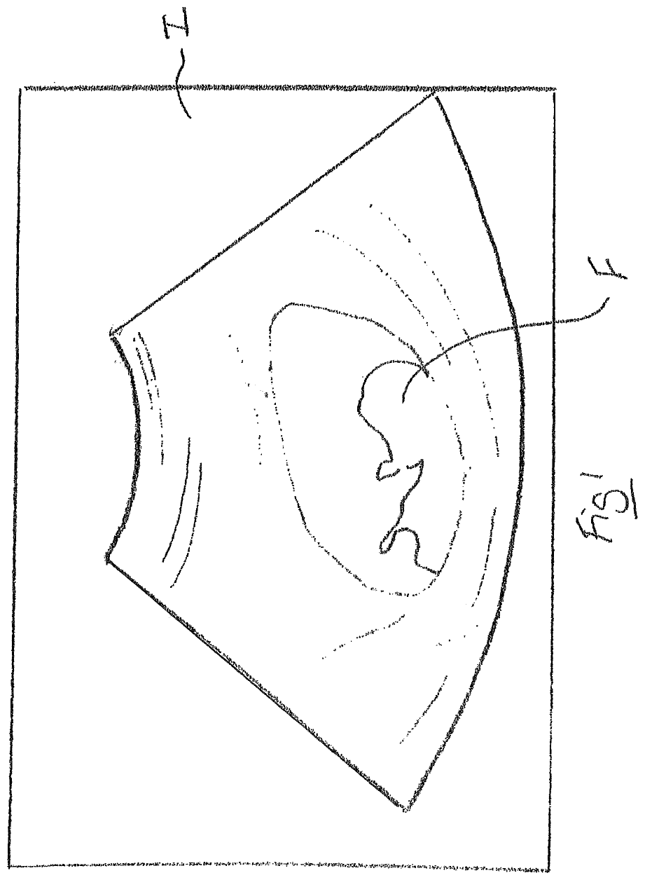

FIG. 1 shows, schematically, an ultrasound image of a human fetus as captured in accordance with an embodiment of the present invention;

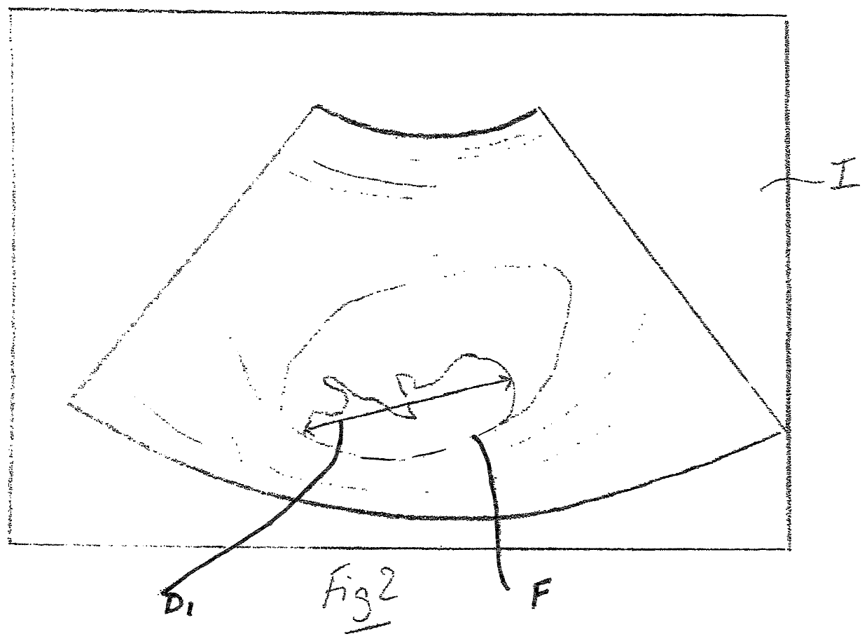

FIG. 2 shows, schematically, the image of FIG. 1, overlaid with dimensional calculations.

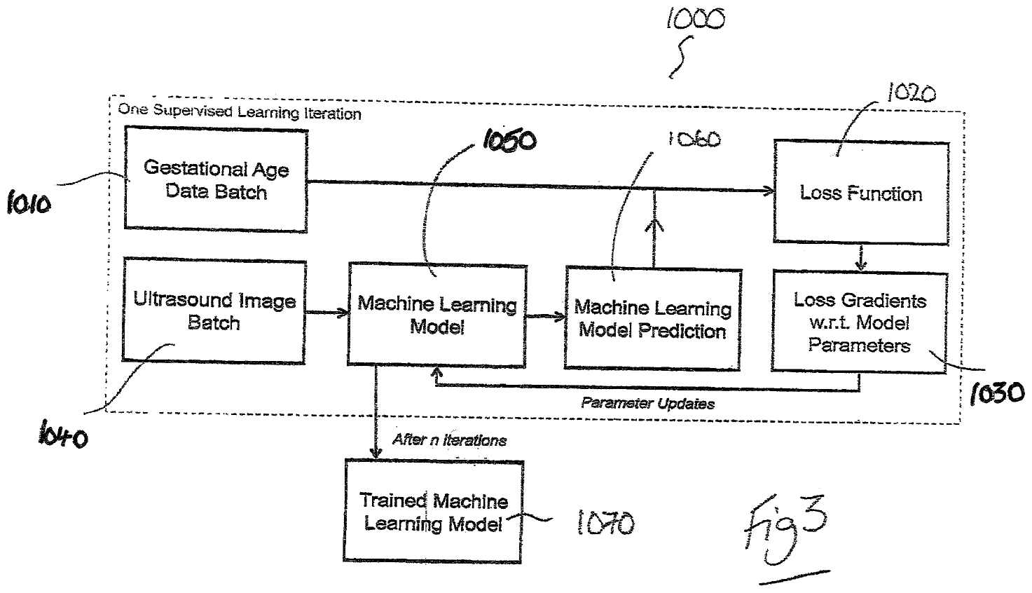

FIG. 3 shows, schematically a method for training a model for use with embodiments of the invention;

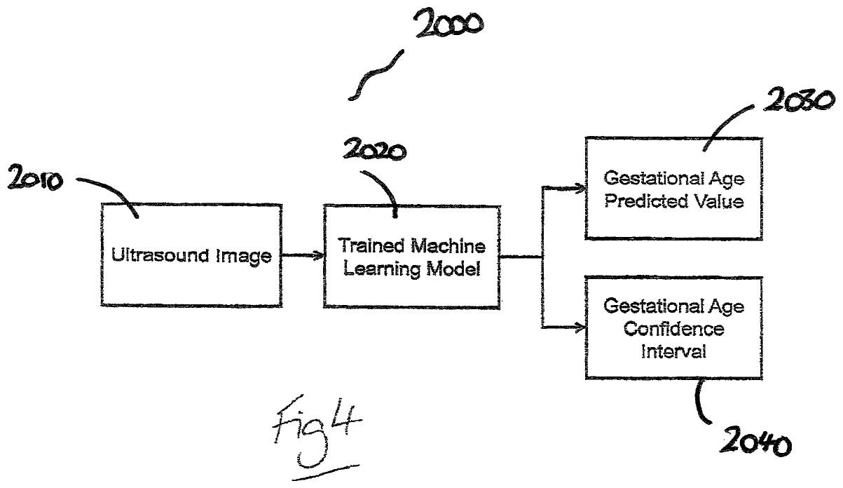

FIG. 4 shows, schematically, an overview of the method according to an embodiment of the invention;

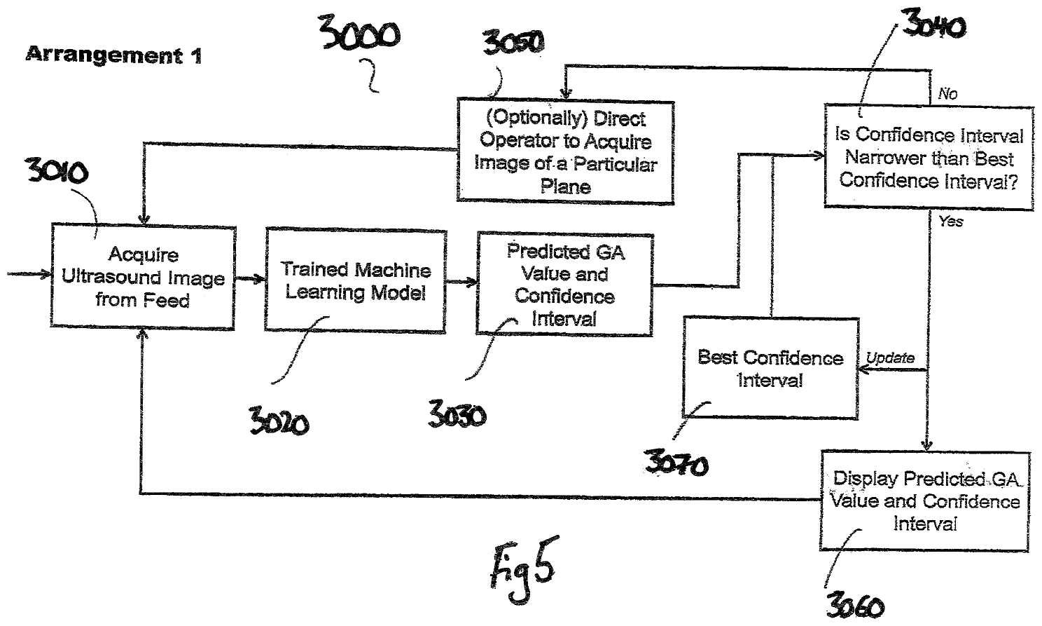

FIG. 5 shows, schematically, a first embodiment of method according to the present invention;

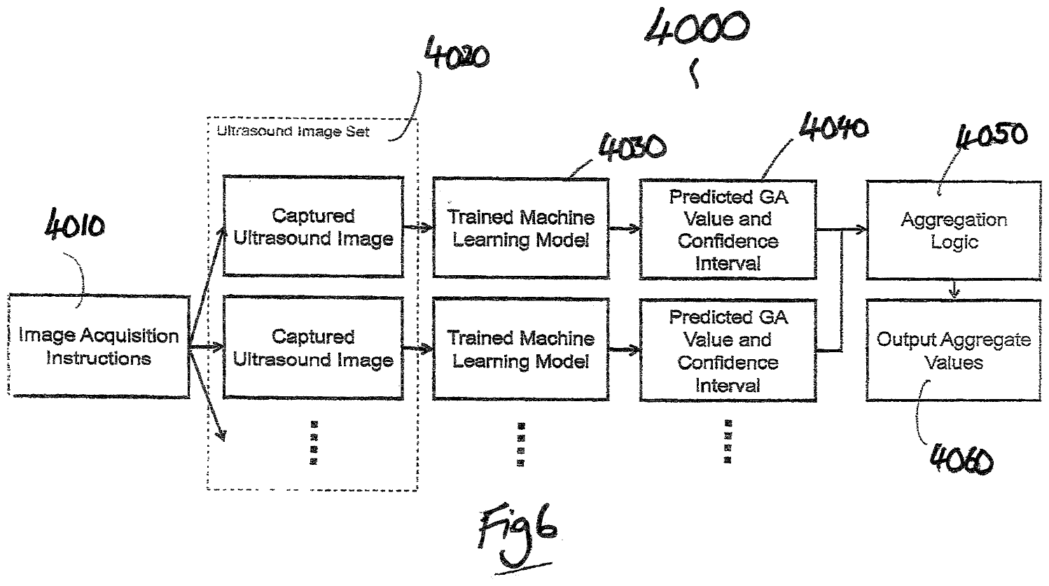

FIG. 6 shows, schematically, a second embodiment of method according to the present invention;

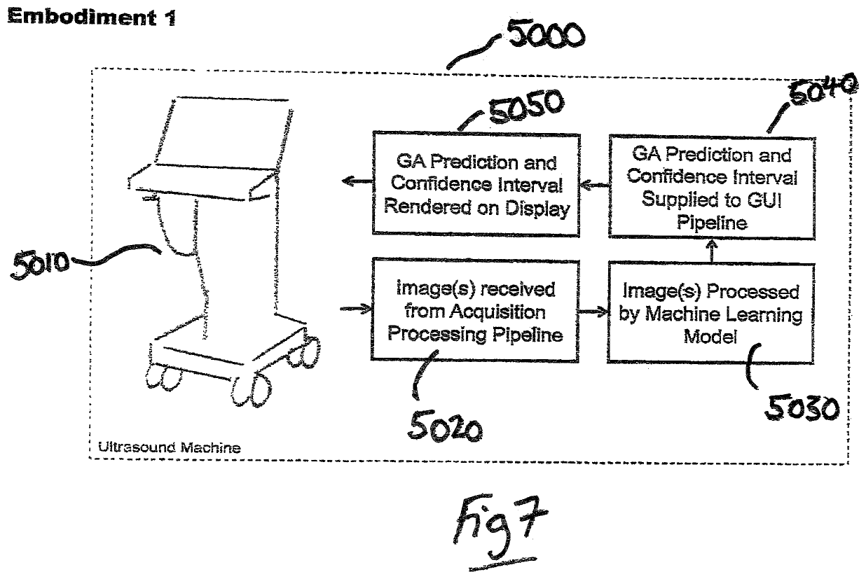

FIG. 7 shows a first embodiment of apparatus according to the present invention;

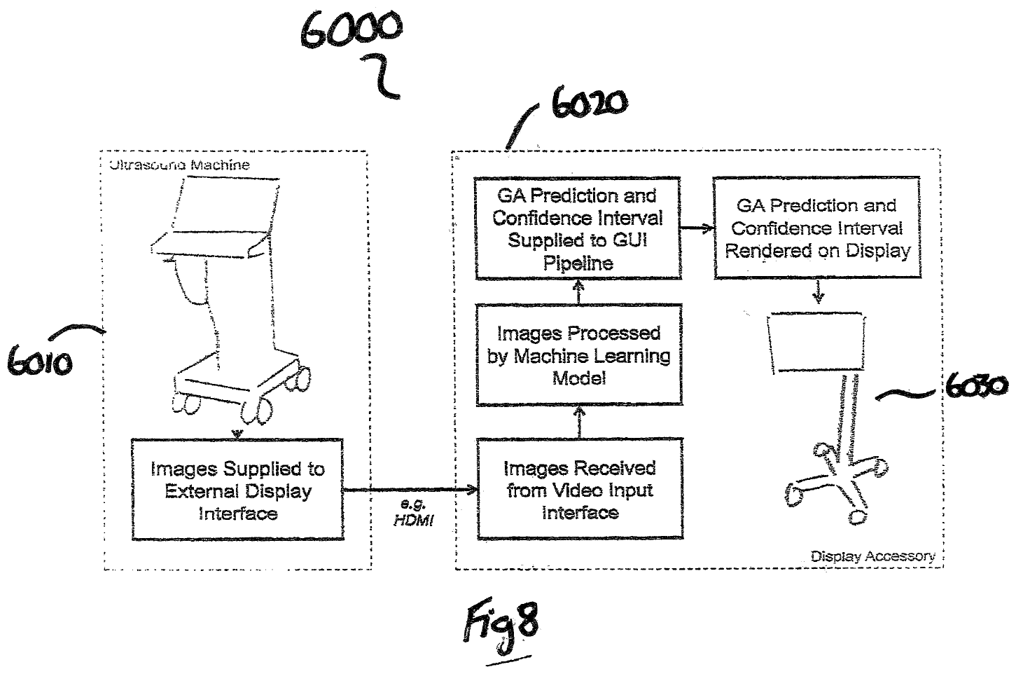

FIG. 8 shows a second embodiment of apparatus according to the present invention; and

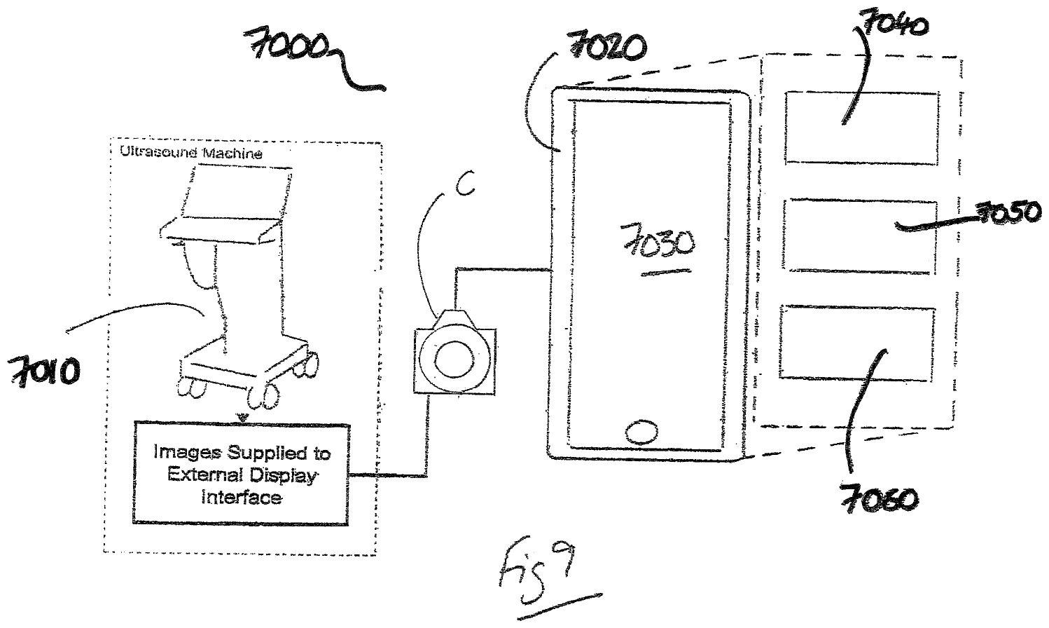

FIG. 9 shows a further embodiment of the present invention.

In the developing fetus, different structures will develop at different rates and will change throughout gestation. In embodiments of the present invention, the way in which various structures change over time, optionally the way in which different structures change with respect to one another, can be used to estimate the age of the fetus using a trained model.

The current standard approach to estimating GA is via obtaining biometric measurements of the fetus and making use of correlations published in the clinical literature between those and GA. These measurements must be taken from imaging planes that have particular properties, and acquiring such images and taking accurate measurements requires a high level of skill from the operator and extensive training. It would be highly beneficial, both to healthcare providers and patients, if it were possible to significantly de-skill the process of accurately estimating GA, such that it could be performed by a much broader population, after minimal training. This would enable assessment to be performed in a primary care setting, for instance. It would be further preferable if such a process could be performed without the operator to look at the ultrasound images at all. This is especially preferable where it is undesirable (or illegal—as in India) to reveal the sex of the fetus.

The required level of operator skill may be reduced somewhat by the use of 3D ultrasound probes which, in principle at least, allows the burden of plane finding and measurement to be shifted somewhat from the operator to an algorithm. This is not a complete shift, since acquiring a suitable 3D volume still requires a higher level of skill than the average person has. Also, in practice, the plane finding and measurement problems are very difficult. All approaches that we are aware of are either less accurate than manual biometric measurement or impractical for use during routine scanning—where outputs are required in near real time—or both. Additionally, 3D ultrasound probes (and the types of ultrasound machines that they can work with) are significantly more expensive than 2D probes. It is therefore desirable to have a method of estimating GA which takes solely 2D ultrasound images as its inputs.

FIG. 1 shows schematically a part of an ultrasound image I of a fetus F obtained in accordance with an embodiment of the present invention.

FIG. 2 illustrates schematically an exemplary dimension D1 which is determined as the image I is processed and which represent distances between chosen points in the image of the fetus F. Using one or more such dimensions, and in particular a consideration of their size relative to the overall image, the processor calculates an estimate of the gestational age (GA) of the fetus, together with an expression of confidence in the prediction, using a machine learning model that has been trained on a previously acquired data set.

In some implementations, embodiments of the present invention use Deep Learning techniques to automatically generate reliable estimates of gestational age from ultrasound images directly, rather than via proxy biometric measurements. The method and associated apparatus are preferably capable of generating accurate estimates from a broad range of images, without having to carefully acquire specific images of the fetus or images having particular properties or even for the operator to have to look at the image. Although the input ultrasound images do not contain information about the real-world scale of the anatomy present within them, they do contain information on the relative scales of different areas of it, which can inform on fetal growth, without requiring information about the real-world size of pixels in the image.

A trained machine learning model is designed to produce an estimate of gestational age from any image of the fetus, rather than requiring images that have certain properties relating to the position of the fetus and presence of certain anatomical features. Since some images presented to the model may be unsuitable for this purpose, the model also reports its confidence in the prediction that it makes, which may be in the form of a prediction interval—or range—optionally containing the actual age with a confidence expressed as a percentage. The prediction intervals are dynamic and based on the suitability of the input image for the task. On good, or highly suitable, images the intervals are narrow, and on bad ones they are wide. The model predictions can then be filtered to include only those which date the gestational age with sufficiently high precision.

In some embodiments of the invention, the sonographer/operative may be directed to obtain certain images of the fetus that are known to be highly predictive of gestational age (e.g., mid-sagittal view of the whole fetus, axial cross-section of the head), and these images combined as inputs to the model to improve its predictive accuracy.

The model outputs may be averaged over a number of images (e.g., a collection selected by the sonographer during scanning, and/or a sample of frames obtained in real-time during scanning) where doing so is found to improve the accuracy of the predictions.

While the primary purpose of the invention is an estimate of gestational age, embodiments thereof may also be capable of estimating the biometric measurements (CRL, head circumference, etc.) that are commonly currently used as the basis of GA estimation and for growth surveillance.

A further purpose of the invention is to additionally output other predictions that are relevant to key aspects of clinical assessment in early pregnancy, including, though not limited to: the presence of multiple fetuses, and in this case their chorionicity and amnionicity; demonstrating fetal viability by confirming cardiac activity and estimating the heart rate of the fetus(es); whether the pregnancy is ectopic; presence of fetal activity; risk of trisomy in the fetus(es); estimated fetal weight and body composition; fetal growth and development including of the brain; presence of pathologies such as acrania, gastroschisis, spina bifida or an anterior wall defect; presence of markers for genetic abnormality such as increased nuchal fold thickness or nasal bone hypoplasia; presence of risk factors for poor outcomes such as stillbirth; the sex of the fetus(es); fetal presentation (e.g. breech, cephalic etc.); placental location; categorisation of placental appearance; estimating the amount of amniotic fluid.

Outputs which depend upon an analysis of multiple frames of video require a small adjustment to model configuration, as follows. Representation vectors, preferably obtained from a layer immediately prior to the prediction heads of the model, should be cached for each frame. The prediction heads should be adjusted to take as input a concatenation of some subset of these cached representation vectors. These adjustments are preferably made during both model training and in its application.

Embodiments of the invention are primarily concerned with the accurate estimation of gestational age during the first trimester of pregnancy without the need for the sonographer to acquire an image of the CRL in perfect position, with the expected result being a substantial reduction in the time required to complete the scan (e.g., ˜7 minutes per scan). However, GA estimates in later stages of pregnancy may also be produced.

Turning to FIG. 3, this shows generally at 1000 a method for training a machine-learning model suitable for use with embodiments of the present invention. At Step 1010, a gestational age (GA) data batch is supplied to a processor which applies a loss function operation at Step 1020. At Step 1030, loss gradients are calculated with respect to selected model parameters. At Step 1050 the data is fed into the machine learning model, along with a batch of corresponding ultrasound images obtained at Step 1040. A machine learning model prediction (ie of the gestational age) is then obtained at Step 1060, before the loop is repeated for n iterations, with parameters updated at each cycle. Finally at Step 1070 the trained machine learning model is complete.

FIG. 4 shows generally at 2000 an overview of a model inference process. At Step 2010 an ultrasound image is obtained. At Step 2020 the image is supplied to the trained machine learning model. At Steps 2030 and 2040, which may be substantially simultaneous, the GA predicted value and corresponding GA confidence interval value are output.

FIG. 5 shows generally at 3000 a first embodiment of method according to the present invention. At Step 3010 an ultrasound image is acquired. The image is fed into the model at Step 3020 and a predicted GA value, together with an associated confidence interval are calculated at Step 3030. At Step 3040 it is determined whether the confidence interval is narrower than a value for the best confidence interval obtained. If not, the ultrasound operation is optionally directed to acquire an ultrasound image of a particular plane in the fetus at Step 3050. On the other hand, if the value is determined to be narrower than the best obtained at Step 3040, the predicted value and confidence interval are displayed at Step 3060 and a register for the value of the best confidence interval is updated at Step 3070.

FIG. 6 shows generally at 4000 a second embodiment of the method according to the present invention. At Step 4010 an ultrasound operator is provided with image acquisition instructions. Next, at Step 4020, a number of ultrasound images are captured by the operative. Each one is provided to the trained model at Step 4030 and predicted GA values together with associated confidence intervals are obtained at Step 4040. The values are aggregated at Step 4050 and the aggregated values are output at Step 4060.

FIG. 7 is a schematic representation of a first embodiment of apparatus according to the present invention. The apparatus is shown generally at 5000. An ultrasound scanning machine 5010 has an image receiving unit 5020 which receives acquired ultrasound images. The images are processed by the model in a processor 5030 and a GA prediction and confidence interval are supplied to a GUI pipeline 5040. A display 5050 of the machine 5010 then displays the prediction and confidence interval.

FIG. 8 shows schematically, generally at 6000 a second embodiment of apparatus according to the present invention. In this embodiment, an ultrasound scanning machine 6010 acquires ultrasound images which are then supplied to an external processing unit 6020, for example via an HDMI or other link. The external unit 6020 receives the images, processes them via the model and outputs a GA estimation and confidence interval which are then rendered on a separate display 6030 of the external unit.

FIG. 9 shows a further embodiment of the present invention, generally at 7000, in which an ultrasound scanning machine 7010 acquires ultrasound images which are then supplied via the camera interface C of a user device 7020, which in this case is a hand-held device such as a cell phone. The user device 7020 is optionally able to display the or each ultrasound image on its display 7030. The processor of the device 7020 uses software, such as may be provided in an app, to perform the tasks of receiving the or each ultrasound image 7040, processing the or each image 7050 and predicting the GA, along with a confidence calculation 7060.

Whereas the embodiments described above use the example of gestational age estimation, it will be understood by the skilled person that there may, as an alternative or in addition, be assessments of different kinds, such as (but not limited to): the presence of multiple fetuses, and in this case their chorionicity and amnionicity; demonstrating fetal viability by confirming cardiac activity and estimating the heart rate of the fetus(es); whether the pregnancy is ectopic; presence of fetal activity; risk of trisomy in the fetus(es); estimated fetal weight and body composition; fetal growth and development including of the brain; presence of pathologies such as acrania, gastroschisis, spina bifida or an anterior wall defect; presence of markers for genetic abnormality such as increased nuchal fold thickness or nasal bone hypoplasia; presence of risk factors for poor outcomes such as stillbirth; the sex of the fetus(es); fetal presentation (e.g. breech, cephalic etc.); placental location; categorisation of placental appearance; estimating the amount of amniotic fluid.

Whilst endeavouring in the foregoing specification to draw attention to those features of the invention believed to be of particular importance, it should be understood that the applicant claims protection in respect of any patentable feature or combination of features referred to herein, and/or shown in the drawings, whether or not particular emphasis has been placed thereon.

1. A method for estimating the gestational age of a fetus, the method comprising obtaining at least one ultrasound image of at least a part of the fetus, and calculating an estimate of the gestational age of the fetus, and a corresponding confidence assessment for the estimate.

2. A method according to claim 1, wherein the gestational age estimate and/or the confidence assessment are produced by a machine learning model.

3. A method according to claim 2, wherein the machine learning model is produced by training on a set of representative ultrasound images of fetuses, for each of which the GA at the time of imaging is known.

4. A method according to claim 2, wherein model training is accomplished via supervised learning, whereby the model is configured to achieve the strongest possible (whilst also robust and generalisable) association between each image and its corresponding GA value.

5. A method according to claim 4, wherein the model and/or supervised training method are additionally configured to output a value range in which the GA is expected to lie.

6. A method according to claim 4, wherein the supervised machine learning process takes the form of deep learning, in which the model is an artificial neural network.

7. A method according to claim 6, wherein the supervised learning method comprises optimising the parameters of the network, via stochastic gradient descent, in order to minimise a loss function.

8. A method according to claim 7, wherein the loss function is constructed such that it generates improved performance for the accuracy of the prediction and the corresponding confidence assessment.

9. A method according to claim 6, wherein the neural network architecture is a convolutional neural network, a vision transformer, or a variant thereof.

10. A method according to claim 1, wherein the method comprises determining at least one relative dimension of one or more anatomical features of the fetus.

11. A method according to claim 1, wherein the method comprises producing a plurality of estimates with a plurality of corresponding confidence values, and filtering the plurality of estimates to select only one or more estimates that meet a predetermined confidence value threshold.

12. A method according to claim 1, wherein the method comprises producing a plurality of estimates, with a plurality of corresponding confidence values, and ranking the estimates based on confidence value.

13. A method according to claim 1, wherein the method includes directing an operative to obtain one or more specific images of the fetus.

14. A method according to claim 13, wherein the operative is instructed to acquire images of certain regions of the fetus that are known (whether through reasoning derived from clinical expertise or via data analysis) to yield accurate estimates of the GA.

15. A method according to claim 13, wherein the operative is dynamically instructed, while scanning, to acquire additional images in the event that the images already acquired have wide confidence ranges associated with them.

16. A method according to claim 1, wherein the method includes arithmetically processing, for example averaging, the estimates of gestational age obtained from a plurality of images.

17. A method according to claim 1, wherein the method comprises a method of estimating biometric measurement(s) including one or more of crown rump length (CRL) and head circumference.

18. A method according to claim 1, wherein the method includes calculating a gestational age estimate and a confidence value from any ultrasound image of the fetus.

19. A method according to claim 1, wherein the method includes calculating a gestational age estimate and/or a confidence value from a fetal image obtained at any stage during gestation.

20. Apparatus for estimating the gestational age of a fetus, the apparatus comprising an ultrasound image capturing device for obtaining an image of at least a part of the fetus, and an electronic processing device arranged to process the image, and to calculate an estimate of the gestational age of the fetus, and a corresponding confidence assessment for the estimate.

21. Apparatus according to claim 20, wherein the processing device is arranged to use a trained machine learning model to calculate the estimate and/or the confidence assessment.

22. Apparatus according to claim 20, wherein the apparatus is configured to provide real-time feedback to the user on the confidence range of the GA estimate that has been obtained, in order that they may direct scanning toward more suitable images when necessary.

23. Apparatus according to claim 20, wherein the processing is implemented directly on the ultrasound apparatus or else on a separate device which either receives a video feed from the ultrasound apparatus or captures a copy of one or more of the ultrasound images via a camera of the device, and optionally displays a result on a separate monitor.

24. A method of making one or more clinical assessments of a fetus, the method comprising obtaining an ultrasound image of the fetus and processing the image using a trained machine learning model to determine one or more of the following, including but not limited to: the presence of multiple fetuses, and in this case their chorionicity and amnionicity; demonstrating fetal viability by confirming cardiac activity and estimating the heart rate of the fetus(es); whether the pregnancy is ectopic; presence of fetal activity; risk of trisomy in the fetus(es); estimated fetal weight and body composition; fetal growth and development including of the brain; presence of pathologies such as acrania, gastroschisis, spina bifida or an anterior wall defect; presence of markers for genetic abnormality such as increased nuchal fold thickness or nasal bone hypoplasia; presence of risk factors for poor outcomes such as stillbirth; the sex of the fetus(es); fetal presentation (e.g. breech, cephalic etc.); placental location; categorisation of placental appearance; estimating the amount of amniotic fluid.

25. Apparatus for making one or more clinical assessments of a fetus, the apparatus comprising a processor for processing the image using a trained machine learning model to determine one or more of the following, including but not limited to: the presence of multiple fetuses, and in this case their chorionicity and amnionicity; demonstrating fetal viability by confirming cardiac activity and estimating the heart rate of the fetus(es); whether the pregnancy is ectopic; presence of fetal activity; risk of trisomy in the fetus(es); estimated fetal weight and body composition; fetal growth and development including of the brain; presence of pathologies such as acrania, gastroschisis, spina bifida or an anterior wall defect; presence of markers for genetic abnormality such as increased nuchal fold thickness or nasal bone hypoplasia; presence of risk factors for poor outcomes such as stillbirth; the sex of the fetus(es); fetal presentation (e.g. breech, cephalic etc.); placental location; categorisation of placental appearance; estimating the amount of amniotic fluid.
